(19) United States
(12) Patent Application Publication
(10) Pub. No.: US 2022/0202852 A1
(43) Pub. Date: Jun. 30, 2022

(54) METHODS AND COMPOSITIONS OF AQUEOUS HYPOBROMOUS ACID FOR THE TREATMENT AND PREVENTION OF INFLAMMATORY CONDITIONS

(71) Applicant: Briotech, Inc., Woodinville, WA (US)

(72) Inventors: Daniel James Terry, Mill Creek, WA (US); Jeffrey Francis Williams, Langley, WA (US)

(21) Appl. No.: 17/565,348

(22) Filed: Dec. 29, 2021

Related U.S. Application Data
(60) Provisional application No. 63/132,429, filed on Dec. 30, 2020.

(51) Int. Cl. A61K 33/00; A61K 9/00; A61P 31/04; A61P 31/10; A61K 9/06

(52) U.S. Cl. CPC A61K 33/00; A61K 9/0014; A61K 9/06; A61P 31/10; A61P 31/04

(57) ABSTRACT

The present disclosure provides methods and compositions of hypobromous acid for use in the treatment and prevention of inflammatory conditions, acute or chronic, of infectious or noninfectious origin, such as those attributable to hypersensitivity mechanisms or trauma in human subjects and animals. The disclosure in various embodiments provides compositions prepared at the time of need for such applications so as to contain pure hypobromous acid formed from stable precursor formulations containing oxidative chlorine in the form of hypochlorous acid, hypochlorite, or other forms of aqueous oxidative chlorine.

### BACKGROUND

### Technical Field

The present disclosure relates generally to methods for treatment of conditions in human subjects and animals that involve inflammation, whether associated with infection or independent of infection. The methods provide for therapy and prevention of microbial infections and the associated tissue responses, as well as for treatment of non-infectious inflammatory conditions, both acute and chronic, that may lead to secondary infections.

### Description of the Related Art

Inflammatory responses may arise in response to invasion of human and animal tissues by microbial agents but can also come about from traumatic disruption of normal tissues, and from allergic reactions, immunologically mediated through several well-established hypersensitivity pathways. Some of those manifest as acute events, such as urticaria, allergic rhinitis, but others, particularly associated with contact, are of the delayed hypersensitivity types and often lead to chronic inflammation. In all cases the classic components of inflammation become evident (pain, swelling, redness, warmth), and usually in the case of allergically mediated processes, the local irritation leads to pruritus.

While there are many therapeutic interventions available for this range of conditions, based on antimicrobial agents (such as antibiotics, antifungal and antiviral compounds), systemic and topical anti-inflammatory steroidal and non-steroidal agents, these are often accompanied by side effects, increasingly important resistance traits in infectious organisms, and prohibitive costs. There is a need for cost-effective, broad spectrum interventions that can both alleviate inflammatory signs, and where necessary, combat infectious microbes, but which do not provoke untoward secondary side effects or encourage resistance. Such interventions will most likely affect one or more of the now well-established inflammatory pathways involving cytokines and other intercellular mediators so as to suppress or modulate the pathological outcomes, and may also directly inactivate infectious organisms when they are present and causal in the disease process.

A need exists to provide a practical means of such interventions, both therapeutically in response to inflammatory disease conditions, and to prevent these arising in response to both infectious and non-infectious triggers of inflammatory events, and to methods and compositions that are safe and effective for prolonged or preventive use, by and on patients and animals of all ages, and states of health, for the purpose of inhibition and control of inflammatory pathological events whether those are infectious in origin or not.

### BRIEF SUMMARY

Briefly stated, the current disclosure is directed towards a method for treating inflammation associated with infectious conditions in a subject. This method includes administering a hypobromous acid composition to an affected area of a human or animal subject in an amount effective to reduce inflammation of the affected area.

In some embodiments of the present disclosure, the affected region is ectodermal, endodermal, or mesodermal. In other embodiments of the present disclosure, the affected region is skin or mucosal epithelium. In another aspect of some embodiments, the hypobromous acid formulation is administered to the affected region from 1 to about 10 times per day. In still another aspect of some embodiments, the hypobromous acid formulation is isotonic, hypotonic, or hypertonic with respect to mammalian body fluids. In such embodiments, the mammalian body fluids include one or more of serum, saliva, tears, vaginal secretions, and serous exudates from body cavity serous epithelia. In yet another aspect of some embodiments, the hypobromous acid formulation is administered in combination with one or more of anti-inflammatory agents, antibiotic, antiviral, antifungal or anti-parasitic agents, and antihistamines.

In some embodiments, the hypobromous acid composition has about 10 to about 1000 ppm of available free bromine and is at least 90% hypobromous acid relative to the total concentration of aqueous bromine species. In another aspect of some embodiments, the hypobromous acid composition is prepared as described herein and administered within six hours of its preparation. In other embodiments, the hypobromous acid composition is prepared as described herein and administered within four hours of its preparation. In still another aspect of some embodiments, the hypobromous acid formulation is prepared at the point of need. In yet another aspect of some embodiments, the hypobromous acid formulation is administered as a solution, a gel, a cream, or a mist. In still other embodiments, the hypobromous acid formulation contains one or more of a viscosity building agent, a surfactant, and a buffering agent.

In another implementation, the current disclosure is directed towards a method for treating inflammation associated with hypersensitivity in a subject. This method includes administering a hypobromous acid composition to an affected area of a human or animal subject in an amount effective to reduce inflammation of the affected area.

### BRIEF DESCRIPTION OF THE SEVERAL VIEWS OF THE DRAWINGS

The foregoing aspects and many of the attendant advantages of this disclosure will become more readily appreciated as the same become better understood by reference to the following detailed description, when taken in conjunction with the accompanying drawings.

FIG. 1 shows the UV-VIS absorbance spectra of representative HOBr solutions adjusted with NaOH to pH 9 useful in the methods of the disclosure.

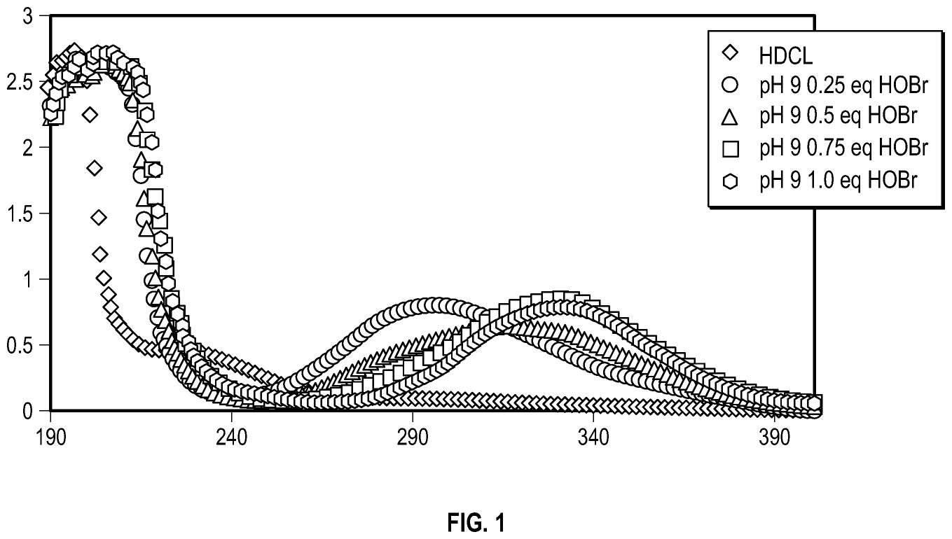

FIG. 2 is a Raman spectrum of a representative HOBr solution useful in the methods of the disclosure.

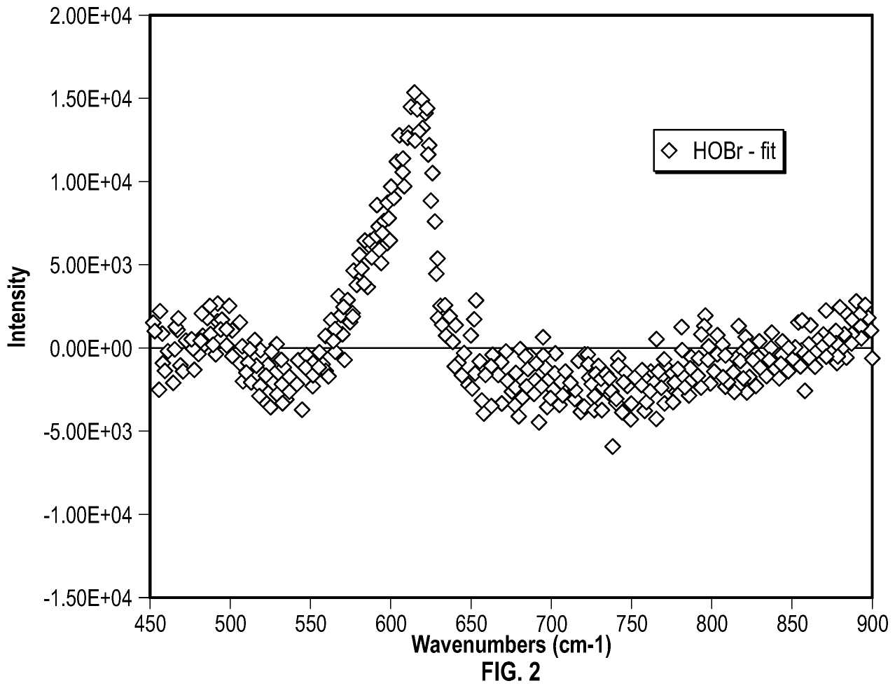

FIGS. 3A-3C compare antimicrobial efficacy comparison of HOBr and HOCl solutions: E. coli, 20 s exposure (3A); S. aureus, 40 s exposure (3B); and A. niger, 40 s exposure (3C). HOBr=diagonal lines; HOCl=stippling.

### DETAILED DESCRIPTION

The disclosure provides methods for treating, preventing, or controlling disease conditions associated with inflammation, including those of infectious origin and those resulting from hypersensitivity mechanisms, acute or chronic, in the absence of pathogenic microbial involvement. The method comprises administering to the affected areas aqueous preparations of hypobromous acid (HOBr), wherein the solution, gel, cream or foam has a content of available free bromine in the range of 10-1000 ppm Br, of which more than 90% is represented by HOBr, and the remainder other aqueous species of Br such as Br-ions, Br₂, and hypobromite ions. The application of these preparations is aimed at reducing and preventing inflammatory symptoms, and reactivation of ongoing inflammatory conditions.

These formulations may be administered topically to affected areas of inflammation or to susceptible sites, including skin, mucous membranes of the ocular, respiratory, reproductive or alimentary tract, via direct deposition, or by means of inhalation, or infusion into the affected or susceptible area. The range of conditions is broad and includes infectious and or allergic involvement of tissues of ectodermal origin, especially skin, eyes, ears, upper respiratory passages (including the nasal passages, pharynx, and larynx), lower respiratory passages (including the trachea, bronchial tree, and lungs), genital mucosae and alimentary tract mucosae, as well as internal surfaces that may be exposed upon deliberate surgical intervention, or as a result of trauma. In the process of administration inflamed sites may be cleansed, and cleared of infectious agents, without resulting in toxicity to the diseased or normal tissues, or other hazards associated with conventional anti-inflammatory measures, such as steroids, antihistamines, antibiotic, antifungal and antiviral entities. These HOBr formulations may also be deployed as adjunctive aids in support of the use of conventional chemotherapeutic measures. They may be used repeatedly over extended periods when necessary without provoking adverse events of any kind.

In certain aspects, the disclosure provides methods of preparation and use of compositions of hypobromous acid for treating, controlling, or preventing inflammatory disease processes, by means of application of the compositions to affected areas, or areas susceptible to inflammation.

Hypobromous acid is an electrophile and an oxidant, produced in the body by enzymatic pathways in response to the recognition of tissue insult or injury by the host innate defense system. It is released into phagocytic vacuoles where it is involved in oxidative attack on invading microorganisms via a variety of chemical reactions, altering substrates including proteins and amino acids, lipids and certain carbohydrates. These chemical reactions are not confined to components of the microbes but can also alter the composition of host cell and extracellular constituents, leading to reaction products with demonstrable anti-inflammatory and antimicrobial properties. These HOBr modified reaction products can contribute in beneficial ways to enhance and accelerate not only control of microbial pathogens, but also the restorative processes that are involved in healing and restoration of normal structure and function of the tissues affected by inflammation. These HOBr formulations are not irritating to normal or disease tissues, do not sensitize these tissues to repeated exposure to HOBr, are not mutagenic, carcinogenic, or cytotoxic. They do not lead to development of resistance to the antimicrobial action of HOBr, even after repeated or prolonged exposure. These formulations are compatible with most but not all conventional antimicrobial therapeutic agents, and other anti-inflammatory chemical measures, so they may be applied in an adjunctive support mode. Used in this manner they may ameliorate some of the side effects and toxicities of conventional anti-inflammatory measures.

Hypobromous acid is a proven powerful biocide, effective against bacterial spores, protozoal trophozoites and cysts, and infectious prion proteins, and is more potent than hypochlorous acid in the inactivation of resistant viruses. Hypobromous acid in aqueous formulations is not stable, and in normal use patterns will degrade at room temperature by dissipation of the active oxidative bromine into bromide ions, and by disproportionation of HOBr into a variety of other bromine species, depending on the pH of the surroundings. Some of these are undesirable, inactive altogether, or will attenuate markedly the efficacy of HOBr in bringing about the beneficial effects of its application. Therefore, the object of this disclosure can be achieved by careful adherence to methods of preparation that enable extremely rapid on-site formation of HOBr within the aqueous medium of the solutions, mists, gels, creams or foams that can be created for practical use. The pKa of HOBr allows for its preponderance as the principal bromine species across an attractively broad range of pH, including those which may be most advantageous for particular formulations, such as tissue adherent gels for example, or for compatibility with prevailing conditions at the targeted site of use, such as in the acidic pH of the vagina, or the neutral pH or mildly alkaline pH of the oral cavity. HOBr may exercise an effect on lipids at certain sites, such as the aural canal, leading to dissolution of waxes and allowing for better penetration of the target tissues. HOBr has known powerful dispersive effects on biofilm, and its deposition at diseased sites which may have allowed for microbial biofilm establishment and therefore offers a convenient and potent means of delivering active antimicrobial agents to the affected tissues, as well as enabling HOBr-mediated enhancement of healing and anti-inflammatory processes. HOBr may alter by bromination certain host proteins such as albumin, so as to permit these proteins to display antiviral properties, thereby inhibiting inflammatory reactions to infectious viral particles.

Hypobromous acid can be produced in the aqueous medium in solutions, gels, creams, and foams by the interaction of bromide ions with active oxidative chlorine, added in appropriate quantities as sodium bromide (NaBr) or potassium bromide (KBr) to Cl-containing precursors of these configurations. By admixture of the stoichiometrically appropriate amounts of bromide with aqueous formulations of oxidative chlorine, preferably HOC1 at pH 3-7, or hypochlorite at pH 7-9, formulations of HOBr can be made to provide concentrations in the range of 10-1000 mg/L of oxidative bromine. Stable precursor aqueous forms of active oxidative chlorine as HOCl or hypochlorite (OCl⁻) can be prepared with suitably long storage shelf lives. These precursor forms may be derived from electrolysis of brine (for HOCl) or from classical chemical methods of making hypochlorite (bleach) solutions, or by other conventionally applied manufacturing methods of producing oxidative chlorine in aqueous form. By a variety of physical means sufficient bromide, as dry granular crystalline particles, as aqueous solutions, or in water-based gels, creams or foams, in amounts equivalent or in slight excess to the quantity of oxidative chlorine present in the precursor, can be added at the point of need to create the advantageously composed formulations of HOBr. In some embodiments, these HOBr formulations may be secured by packaging, both for professional and consumer use. For example, in one embodiment a pack is provided for the end user to make HOBr by mixing parts of a formulation, even if the end user does not know that HOBr is being made. In this regard, the end user could be a consumer without any specialized training, an employee at a pharmacy, an emergency response field technician, or a medical profession at any type of medical facility. These formulations will then provide for their use, preferably within four hours for optimum performance, preferably after no more than six hours, as HOBr preparations for topical applications to affected sites. Those sites will be primarily of ectodermal origin, but in certain circumstances they may be applied as anti-infective and anti-inflammatory agents to internal tissues of endodermal or mesodermal origin.

It will be appreciated that the present disclosure provides for production of HOBr in situ at the point of need by the controlled interaction of HOCl or OCl⁻ or both with a source of bromine (e.g., NaBr or KBr). The superior oxidizing position of chlorine over bromine leads to formation of Br⁺¹ and over a wide range of pH (3-9) leads to an immediate formation of HOBr. Disproportionation and decay of HOBr leads within hours of its formation to other aqueous bromine species: some completely inactive (Br), some much less active than HOBr, and some with unattractive toxicity compared to HOBr. As a consequence, in the methods of the disclosure, HOBr is produced in pure form and used within about 6 hours, preferably within about 4 hours of its production, before these changes bring about deleterious alterations in the composition. The addition of bromide (e.g., NaBr or KBr) can be in the form of solid crystals, solutions in water, gels, etc., with the amounts to be added predetermined to match the desired amount of Br⁺¹ to be created in the composition. These additions can conveniently designed to depend on a user action, whereby, for example, solutions in two separate chambers of the product are mixed at the point of use, or solid crystals of bromide are made to fall into the stable chlorine solution on opening the container, or by mixing two preparations of stable gel. Stable solutions of electrolytically produced HOCl can be used as the preconfigured formulation. Hypochlorite can also be used. Bromide, whether in solution or as a solid, or in a gel, is stable under all conditions and stresses. All such compositions as outcomes of the chemical reaction can be used to impact topically the inflammatory events arising as described above.

The preparation and characterization of representative useful HOBr compositions is described in Examples 1-6. The preparation of a representative aqueous solution is described in Example 1; a representative aqueous gel is described in Example 2, and a representative aqueous cream is described in Example 3. The efficacy of representative HOBr compositions is described in Example 4 and 5. The safety of a representative HOBr compositions is described in Example 6.

### Representative HOBr Compositions

The methods of the disclosure utilize hypobromous acid compositions. In certain embodiments, the hypobromous acid compositions is a bufferless, electrolyzed, hypobromous acid composition comprising a hypobromous acid and a chloride salt.

In certain embodiments, the hypobromous acid composition is an aqueous hypobromous acid composition having a hypobromous acid concentration from about 10 to about 300 mg/L, a pH from about 3 to about 8.5, an oxidative reduction potential (ORP) of about +1000 millivolts, and containing from about 0.85% to about 2.0% by weight chloride salt based on the total weight of the composition.

In other embodiments, the hypobromous acid composition is an aqueous hypobromous acid composition having a hypobromous acid concentration from about 5 to about 350 mg/L, a pH of about 7 to about 8, an oxidative reduction potential (ORP) of about +900 millivolts, and containing from about 0.85% to about 2.0% by weight chloride salt based on the total weight of the composition.

In certain of these embodiments, the chloride salt is an aqueous soluble chloride salt selected from sodium chloride, potassium chloride, magnesium chloride, and ammonium chloride. In certain embodiments, the chloride salt is sodium chloride. In certain embodiments, the composition contains about 2.0% by weight chloride salt based on the total weight of the composition. In certain embodiments, the composition contains about 2.0% by weight sodium chloride based on the total weight of the composition.

The composition does not contain aqueous oxidative bromine in the form of HOBr to an extent less than 90%. As used herein, “oxidative bromine” refers to all oxidizing bromine species (e.g., HOBr, molecular bromine, bromate, bromite, hypobromite) detectable by, for example, repetitive-scan Raman spectroscopy. In certain embodiments, the composition includes <200 ppm aqueous oxidative bromine. In other embodiments, the composition includes <100 ppm aqueous oxidative bromine. In further embodiments, the composition includes <50 ppm aqueous oxidative bromine. It will be appreciated that for HOBr solutions, the composition does not contain less than 90% aqueous oxidative bromine in the form of HOBr detectable by, for example, repetitive-scan Raman spectroscopy or spectrophotometric analysis in the UV range. In some embodiments, other than the hypohalous acid, the electrolyzed, hypohalous acid composition does not contain a detectable amount of aqueous oxidative chlorine or aqueous oxidative bromine as determined by Raman Spectroscopy.

### Use of HOBr Compositions for Treatment of Inflammatory Conditions

Applications of such HOBr compositions can be used in therapy of inflammatory conditions, whether caused by infectious agents, allergic hypersensitivity reactions of types 1, 2 or 3, by or physical injury, or as used adjuncts to conventional anti-infective or anti-inflammatory measures, or as preventive interventions prior to potential exposure to provocative agents such as allergens or irritants. The solutions may contain in addition to HOBr concentrations of sodium chloride (NaCl) such as to make the formulations isotonic with mammalian cells, or hypotonic or hypertonic, depending on the most appropriate fit with the targeted site. Similarly, the pH of the compositions may be modified by the incorporation of buffers, suitable to maintain the appropriate pH for the targeted site, such as pH 4 for vaginal mucosa, slightly acidic for the conjunctiva, or neutral to slightly alkaline for oral use. Generally, buffering within the range of pH 3-9 will ensure preponderance of HOBr to the exclusion of all other aqueous species for the prescribed period of four hours after its formation in the manner prescribed by this disclosure. HOBr may be stabilized chemically in aqueous medium with heterocyclic additives but this is not advisable because their presence will cause deviations from the beneficial profile of HOBr in pure condition in respect of both safety and efficacy.

The antimicrobial efficacy of HOBr in the compositions described herein extends across a broad range of infectious agents, usually impacting their infectivity in short contact times, usually measured in seconds, but always tempered by the presence of host-derived substrates that can be oxidized or brominated. However, the reaction products of these interactions of HOBr with cellular and extracellular constituents can themselves display powerful antimicrobial and anti-inflammatory properties. Examples are N-bromotaurine, N-bromotryptophan, and other brominated aminoacids, intact polypeptides and proteins, as well as bromohydrins, bromoflavenols, and the like. HOBr-modified cellular and extracellular substrates have been shown to be more effective in resolving certain infectious inflammatory conditions than those modified by HOCl. By these mechanisms the beneficial effects of topical applications of compositions containing HOBr are perpetuated so as to bring about relief from the inflammatory consequences of infectious, allergic, traumatic events, especially at dermal and mucosal surfaces. Those consequences typically prevented or attenuated by HOBr compositions include pain, itching (pruritus), erythema, induration, and excessive local production of heat.

Inflammatory conditions resulting from Type I hypersensitivity, provoked by exposure to an allergen, such as a pollen or dander, can be beneficially treated with compositions of HOBr described herein. Allergens cross-link the bound Immunoglobulin E on sensitized mast cells, resulting in degranulation and the secretion of pharmacologically active mediators such as histamine, leukotrienes, cytokines and prostaglandins. These mediators cause vasodilation and smooth-muscle contraction, such as in allergic asthma, allergic conjunctivitis, allergic rhinitis, anaphylaxis, angioedema, urticaria, eosinophilia, drug allergy (e.g., penicillin, cephalosporin), or food allergy. Hypobromous acid inhibits the release of these mediators from mast cells at epithelial surfaces and can thereby prevent inflammatory outcomes from exposure to allergens.

In other kinds of hypersensitivity, such as Type 2, foreign allergens adhere to host cell surfaces and are recognized by the immune system, which may launch an attack on the now abnormally modified host cells. These attacks trigger the release of mediators that cause inflammation and can be counteracted by the presence of HOBr or by reaction products of its interaction with host substrates. The accumulation of antigens and host-produced antibodies can in certain well-defined circumstances also trigger inflammatory mediator release, recognized as Type 3 hypersensitivity.

In Type 4 hypersensitivity (so-called delayed-type hypersensitivity) inflammation is not antibody-mediated but depends on cell-mediated responses, leading to release of interleukins (such as IL-1β, IL-2, IL-4, IL-6, IL-8, IL-18), TNF-α,and interferon, accompanied often by the production of tissue damaging enzymes that cause long term, persistent inflammation. Allergic contact dermatitis is typical of Type 4 hypersensitivity, and results from contact with environmental chemicals such as poison ivy and nickel, and leads to protracted itching, that in turn can lead to scratching and secondary infections. Inflammation is then generated by both non-infectious means, and complicated by superimposed infections. Compositions of HOBr as described herein can advantageously inhibit inflammation, and also attack infectious agents in the lesions. HOBr can also inhibit tissue metalloprotease enzymes generated during inflammation and responsible for extending and prolonging tissue damage.

Diseases caused by prolonged and/or untreated inflammation, such as radiation dermatitis, can also be beneficially managed by use of HOBr compositions topically. Infectious inflammatory conditions that may be treated in accordance with the disclosure affect a wide range of skin and mucosal systems, and therefore include conjunctivitis, blepharitis, uveitis, rhinitis, sinusitis, stomatitis, eczema, acne, psoriasis, bacterial and fungal vaginitis, and Hidradenitis suppurativa, among many other clinically recognizable ailments that involve acute and chronic inflammatory processes accompanying infection. Where these conditions involve biofilm, HOBr compositions can disperse the polymeric matrix of the biofilm, to facilitate wound cleansing, bringing about more effective removal of pathogens and debris.

In other circumstances the HOBr composition can be administered prophylactically, such as where for example eye infections are likely to occur or be transmitted among children in close contact with one another. The disclosure therefore can involve use of the compositions in routine cleaning of the eyes and surrounding areas, such as the eyelids, or routine rinsing of contact lenses to decontaminate them and clear them of debris and biofilm. HOBr is known to have efficacy against ameobic protozoa that can be causal in chronic inflammatory disease of the eye. The compositions can be applied to the environment via fogging, misting, or humidifying with a vapor so as to prevent the transfer of pathogens from air droplets into the eyes or respiratory passages of exposed individuals.

The HOBr compositions of the present disclosure may be administered in any appropriate dosage form such as a liquid, aerosol, gel, cream, or other types of compositions.

The following examples are provided for the purpose of illustrating, not limiting, the disclosure.

### EXAMPLES

The following examples are put forth to provide those of skill in the art with a complete account of the new methods of preparation and use of pure hypobromous acid, and the means of detection and measurement of the effectiveness of these solutions. The examples are not intended to limit the scope of what the inventors regard as the disclosure, nor do they represent all the experiments that have been done to demonstrate the utility of the methods and compositions disclosed herein.

### Materials and Methods

BrioHOCl™ was supplied by Briotech Inc., Woodinville, Wash. Briefly, HOCl results from electrolysis of an aqueous solution of sodium chloride so as to provide at the anode conditions that attract and stabilize reaction products that form HOCl. The end-product is a solution with a range of pH on packaging and storage of 4-6 at warehouse environmental temperatures (3.5° C. to 35° C.), an ORP of 1100+, a salt (NaCl) concentration of either 0.9% or 1.8%, and a free chlorine concentration of 250-300 mg/L at the time of production.

Hypobromous acid (HOBr) was prepared by the exposure of a slight excess of one equivalent of aqueous bromide ion (as NaBr) to one equivalent of HOCl. This solution was prepared fresh for use in tests for inactivation of resistant microbial organisms.

### Active Chlorine or Bromine Measurement

Hach reagent kits for Total Halogen (Hach Company, Loveland, Colo.) were used for determination of the active Cl or Br content of formulations.

### UV/Vis Spectrophotometry

Test solutions of HOBr and HOCl were loaded into 1 mL quartz cuvettes, and spectra obtained using a BioMate 3S UV-Visible Spectrophotometer. The instrument was blanked using Nanopure water, and test solutions consisted of undiluted BrioHOCl™ at selected time points in the sequential sampling of product stored at room temperature. Absorbance was measured from 190 to 400 nm, with peak absorbance for HOBr showed an absorbance peak at 260 nm, while HOCl registered at 238 nm.

### Raman Spectroscopy

Spectra were obtained using a Renishaw InVia Raman microscope. Spectra were observed using an excitation wavelength of 785 nm with undiluted BrioHOBr™ in a 1 mL quartz cuvette. The acquisition time for each scan was 20 seconds, and 100 acquisitions were accumulated. A deionized water blank was scanned in the same manner and subtracted from the test sample data using Igor software.

### Example 1

### Formation of Aqueous Solutions of HOBR From Stable Solutions Containing HOCL

In a test for a procedure that would allow for the formation of an HOBr solution from a solution of BrioHOCl of known Cl⁺¹ concentration, a slight excess of an equivalent of sodium bromide crystals was added followed by vigorous shaking. The conversion of HOCl to HOBr proceeded very rapidly such that in a few tens of seconds HOCl was no longer detectable spectroscopically, whereas a new peak of HOBr became evident at 260 nm. By adjusting the pH upwards this HOBr was instantly converted to OBr⁻ which exhibits a characteristic peak in the U/V range at 330 nm.

### Example 2

### Formation of HOBR in Aqueous Gel

To an aqueous gel consisting of 3% Laponite smectite clay particles allowed to swell in water and then mixed with a solution of HOCl at 300 ppm, pH 5.8, a slight excess of an equivalent (e.g., within ten percent over an equivalent) of NaBr was added with brisk mixing. Within several minutes the UV-Vis spectrophotometric profile showed conversion of the HOCl to HOBr, and a new peak of absorption at 260 nm.

### Example 3

### Formation of HOBR in an Aqueous Cream

To an aqueous cream preparation consisting of 16% Veegum smectite clay allowed to swell at room temperature overnight, an equal volume of a solution of HOCl at 300 ppm, pH 5.8 was added. The resulting cream was mixed with an equal volume of a similar cream containing 150 mg/L of NaBr. Within minutes the cream showed the appearance of a new peak of absorption at 260 nm by UV-Vis spectroscopy, indicating the formation of HOBr, and the complete disappearance of HOCl.

### Example 4

### Efficacy of HOBR in Inactivation of Resistant Spores

Test samples of freshly prepared HOBr containing no detectable HOCl by UV-Vis spectroscopy showed high degrees of efficacy in inactivating spores of Bacillus subtilis. Exposures as brief as 20 seconds to HOBr at approximately 25 ppm were sufficient to produce LRV of 6. In the same experimental protocol HOC1 at 230 ppm was required to produce 6 LRV in the same contact time. As soon as the HOCl concentration used was below 230 ppm, the LRVs fell into the 2-4 range. At 25 ppm of HOCl there was no detectable effect on Bacillus spores in 20 seconds of contact.

In Raman spectroscopy of these preparations there was no peak corresponding to HOCl, but a new peak appeared at wavenumber 615/cm attributable to HOBr.

### Example 5

### Comparative Efficacy of HOBR And HOCL Vs Bacterial and Fungal Pathogens

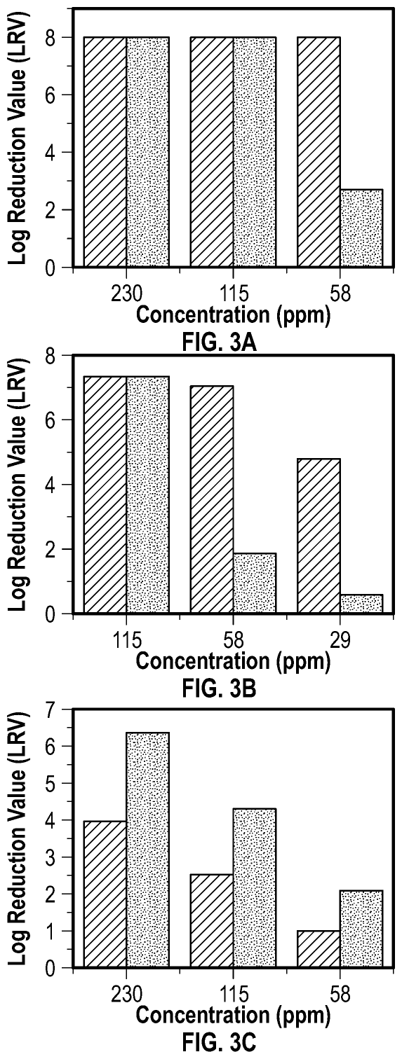

Preparations of HOCl stored at 22° C. for several weeks and freshly prepared HOBr solutions were compared in their antimicrobial properties versus suspensions of E. coli, S. aureus, and A. niger (see FIG. 3). At high concentrations (≥115 ppm) HOBr and HOCl were equally effective at inactivating E. coli and S. aureus. At lower concentrations HOBr was markedly more effective than HOCl against these organisms. However, HOCl was equally or more effective than HOBr against A. niger at all concentrations tested.

### Example 6

### Safety of Topically Applied Freshly Prepared Aqueous HOBR

A solution of HOBr was made by addition of an equivalent of NaBr to a solution of HOCl containing 200 ppm of Cl. This solution was also applied to human skin and mucous membranes, including conjunctiva, without any indication of adverse effects on these epithelial surfaces.

While illustrative embodiments have been illustrated and described, it will be appreciated that various changes can be made therein without departing from the spirit and scope of the disclosure.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for treating inflammation associated with infectious conditions in a subject, comprising administering a hypobromous acid composition to an affected area of a human or animal subject in an amount effective to reduce inflammation of the affected area.

2. The method of claim 1, wherein the affected area is ectodermal, endodermal, mesodermal, skin, or mucosal epithelium.

3. The method of claim 1, wherein the hypobromous acid formulation is administered to the affected area from 1 to about 10 times per day.

4. The method of claim 1, wherein the hypobromous acid formulation is isotonic, hypotonic, or hypertonic with respect to mammalian body fluids.

5. The method of claim 4, wherein the mammalian body fluids include one or more of serum, saliva, tears, vaginal secretions, and serous exudates from body cavity serous epithelia.

6. The method of claim 1, wherein the hypobromous acid formulation is administered in combination with one or more of anti-inflammatory agents, antibiotic, antiviral, antifungal agents, anti-parasitic agents, and antihistamines.

7. The method of claim 1, wherein the hypobromous acid composition has from about 10 to about 1000 ppm of available free bromine and is at least 90% hypobromous acid relative to the total concentration of aqueous bromine species.

8. The method of claim 1, wherein the hypobromous acid composition is prepared and administered within six hours of its preparation, prepared and administered within four hours of its preparation, or prepared and administered at a time when the hypobromous acid composition is needed.

9. The method of claim 1, wherein the hypobromous acid formulation is administered as a solution, a gel, a cream, or a mist.

10. The method of claim 1, wherein the hypobromous acid formulation contains one or more of a viscosity building agent, a surfactant, and a buffering agent.

11. The method of claim 1, wherein the inflammation of the affected area comprises infectious or allergic involvement of tissues, including one or more of skin, eyes, ears, nasal passages, pharynx, larynx, trachea, bronchial tree, lungs, genital mucosae, and alimentary tract mucosae.

12. The method of claim 1, wherein the hypobromous acid formulation is administered to affected areas of inflammation, without resulting in irritation or toxicity to the affected area.

13. The method of claim 1, wherein the hypobromous acid formulation is administered, via inhalation, to affected areas of inflammation, including the respiratory tract.

14. A method for treating inflammation associated with hypersensitivity in a subject, comprising administering a hypobromous acid composition to an affected area of a human or animal subject in an amount effective to reduce inflammation of the affected area.

15. The method of claim 14, wherein the affected area is ectodermal, endodermal, mesodermal, skin, or mucosal epithelium.

16. The method of claim 14, wherein the hypobromous acid formulation is administered to the affected region from 1 to about 10 times per day.

17. The method of claim 14, wherein the hypobromous acid formulation is isotonic, hypotonic, or hypertonic with respect to mammalian body fluids.

18. The method of claim 17, wherein the mammalian body fluids include one or more of serum, saliva, tears, vaginal secretions, and serous exudates from body cavity serous epithelia.

19. The method of claim 14, wherein the hypobromous acid formulation is administered in combination with one or more of anti-inflammatory agents, antibiotic, antiviral, antifungal agents, anti-parasitic agents, and antihistamines.

20. The method of claim 14, wherein the hypobromous acid composition has from about 10 to about 1000 ppm of available free bromine and is at least 90% hypobromous acid relative to the total concentration of aqueous bromine species.

21. The method of claim 14, wherein the hypobromous acid composition is prepared and administered within six hours of its preparation, prepared and administered within four hours of its preparation, or prepared and administered at a time when the hypobromous acid composition is needed.

22. The method of claim 14, wherein the hypobromous acid formulation is administered as a solution, a gel, a cream, or a mist.

23. The method of claim 14, wherein the hypobromous acid formulation contains one or more of a viscosity building agent, a surfactant, and a buffering agent.

24. The method of claim 14, wherein the inflammation of the affected area comprises infectious or allergic involvement of tissues, including one or more of skin, eyes, ears, nasal passages, pharynx, larynx, trachea, bronchial tree, lungs, genital mucosae, and alimentary tract mucosae.

25. The method of claim 14, wherein the hypobromous acid formulation is administered to affected areas of inflammation, without resulting in irritation or toxicity to the affected area.

26. The method of claim 14, wherein the hypobromous acid formulation is administered, via inhalation, to affected areas of inflammation, including the respiratory tract.
